(19) United States
(12) Patent Application Publication
(10) Pub. No.: US 2026/0007771 A1
(43) Pub. Date: Jan. 8, 2026

(54) VETERINARY COMPOSITIONS

(71) Applicant: Carus Animal Health Limited, Reigate (GB)

(72) Inventors: Gregory James FIRTH, Sheffield (GB); Rajesh Muru ODEDRA, Stevenage (GB)

(21) Appl. No.: 19/257,882

(22) Filed: Jul. 2, 2025

(30) Foreign Application Priority Data
Jul. 3, 2024 (GB) 2409634.9

(51) Int. Cl. A61K 47/69; A61K 9/12; A61K 47/12; A61K 47/26; A61K 47/64; A61P 31/04

(52) U.S. Cl. CPC A61K 47/6925; A61K 9/122; A61K 47/12; A61K 47/26; A61K 47/6455; A61P 31/04

(57) ABSTRACT

The present disclosure relates to veterinary compositions for the treatment and/or prevention of mastitis in cattle, as well as to methods of treating and/or preventing mastitis in cattle.

### CROSS-REFERENCE TO RELATED APPLICATIONS

This application claims the benefit of priority to UK Patent Application No. 2409634.9, filed Jul. 3, 2024, which is incorporated by reference in its entirety.

### FIELD

The present disclosure relates to veterinary compositions for the treatment and/or prevention of mastitis in cattle, as well as to methods of treating and/or preventing mastitis in cattle.

### INTRODUCTION

Clinical mastitis is an inflammatory condition of the mammary gland typically caused by bacterial infection. In dairy cows it can result in swelling, redness, heat, and pain in the udder leading to production of poor-quality milk and reduction of yield and, in severe cases, septicaemia and death. In contrast, subclinical mastitis is mammary disease which does not result in visible inflammation and is largely asymptomatic other than causing reduction in milk yield and elevation of somatic cell count. Subclinical mastitis is detected by the routine measurement of somatic cell counts in milk samples, the accepted upper limit for infection-free glands being 200,000 cells per millilitre.

Clinical mastitis is usually seen in lactating cows. Nonetheless, despite innate protection of each udder quarter provided by lactoferrin, hydrolytic enzymes, and immunoglobulins in mammary secretions, as well as the keratin plug in the streak canal, the majority of infections detected in lactating cows are acquired during the preceding dry period. Indeed, each udder quarter is at greatest risk of infection in the early dry period when the gland is still undergoing involution and a keratin plug has not yet formed. Results from a study by Dingwell et. al (Prev Vet Med. 2004 Apr. 30; 63 (1-2): 75-89. doi: 10.1016/j.prevetmed.2004.01. 012. PMID: 15099718, incorporated herein by reference in its entirety) suggested 47% of teats are without a keratin plug one week after dry off and 23% remain open even after 6 weeks. In these udder quarters, therefore, a physical barrier preventing ingress of bacteria into the gland does not exist. Although secreted proteins largely limit pathogen multiplication and prevent manifestation of clinical mastitis in the dry period, they are unable to prevent the establishment of quiescent infection which can become fulminant during the subsequent period of lactation.

Historically, blanket dry cow therapy was employed to counteract infection, in which long acting intramammary antibiotic products were routinely employed at dry off to eliminate subclinical infections and to prevent new infections from being acquired during the ensuing dry period. Thus, the antibiotic products were used both for therapy and prophylaxis. However, due to concerns regarding antimicrobial resistance and the requirement for appropriate antibiotic stewardship, the indiscriminate use of broad-spectrum antibiotics has been discontinued. Currently, intra mammary antibiotics are permitted at dry off in those udder quarters known to be infected, identified as a result of evaluation of somatic cell count and by microbiological assessment, but in non-infected udder quarters alternative means of protection are required.

The realization that many teats remained open without a functional keratin plug during the early dry period led to the development of teat sealants to provide protection for non-infected quarters in which antibiotic administration is not permitted. There are two types of teat sealants: external teat sealants encompassing materials that adhere to the external surface of the teat and internal teat sealants comprising inert formulations of bismuth salts. External teat sealants have a poor retention and thus need to be replaced regularly, and as a result, they are not widely used. In contrast, internal teat sealants have been widely adopted and have become an integral component of dairy cow management in the dry period.

Cao et al (J. Dairy Sci. 90:3980-3985, 2007) describe the administration of nisin, an antimicrobial peptide, to lactating dairy cows with clinically mastitic quarters. The results were similar to those obtained from gentamicin which was used as a comparator in the trial. Huang et al (J. Dairy Science. 105:3530-3543) have reported that nisin K exerted an anti-inflammatory effect by inhibiting the ERK1/2 and p38 MAPK signaling pathway and promoting the blood-milk barrier on liposaccharide (LPS)-induced mastitis.

Galvao et al (Applied and Environmental Microbiology 86 18 e1066-20, 2020) compared the administration of microparticles containing chitosan to the uterus of cows to the administration of ceftiofur and reported that “our study indicates with a high degree of confidence that chitosan microparticles, tested as described here, are detrimental to uterine health.”

EP3265139B describes a self-assembled microparticle comprising (i) an acid of formula HOOC—(CH₂) n-COOH wherein n is at least 5 and not more than 40 and (ii) an organic base in which the molar ratio of acid groups to basic groups is from 0.6 to 1.4:1, the microparticle being obtainable by a process comprising contacting the acid and base in aqueous solution, wherein the acid is insoluble in the aqueous solution and the organic base is soluble in the aqueous solution, and wherein the microparticle comprises a multi-lamellar structure.

Bismuth subnitrate is a commonly used internal teat sealant. However, it is a recognised risk of the use of bismuth subnitrate that pathogens may be inadvertently introduced into the mammary gland. In addition, bismuth subnitrate is carried over into milk when the cow's dry period ends. Therefore, there is a need for an internal teat sealant which minimizes these issues.

### BRIEF DESCRIPTION OF THE FIGURES

FIG. 1 compares the log reduction in bacterial biofilm cell concentration which is observed upon addition of sterile water (the control formulation), a suspension containing microparticles without surface-bound ε-polylysine, and a suspension containing microparticles with surface-bound ε-polylysine.

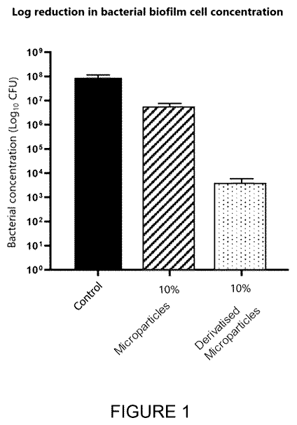

### SUMMARY

The present disclosure provides compositions and methods for treating and/or preventing mastitis in cattle by sealing the animal's teats with microparticles that have antimicrobial activity. In some embodiments, the antimicrobial microparticles remain in situ in the lowest part of the teat in the vicinity of streak canal. In some embodiments, the compositions and methods described herein prevent intra mammary infections in cows during their dry period.

In some embodiments, the disclosure provides a method for treating or preventing mastitis in cattle, comprising administering to the cattle an effective amount of a suspension comprising a plurality of first microparticles at a concentration of 5% to 60% w/v, wherein the first microparticles are Derivatized Microparticles comprising (i) a veterinary acceptable saturated or unsaturated di-carboxylic acid comprising 8 to 20 carbon atoms, (ii) an organic polycationic polymer, and (iii) an organic polycationic polymer bound to a surface of the first microparticles.

In some embodiments, the suspension is administered during a cow's dry period.

In some embodiments, the suspension further comprises a plurality of second microparticles, wherein the second microparticles are Microparticles comprising (i) a veterinary acceptable saturated or unsaturated di-carboxylic acid comprising 8 to 20 carbon atoms and (ii) an organic polycationic polymer, wherein the total concentration of first and second microparticles in the suspension is 5% to 60% w/v. In some embodiments, the ratio of Derivatized Microparticles to Microparticles in the suspension is 90:10 to 10:90 w/w.

In some embodiments, the suspension is antimicrobial.

In some embodiments, the one or more of the polycationic polymers in the first and/or second microparticles (i.e., the Derivatized Microparticles and/or the Microparticles) is/are antimicrobial.

In some embodiments the Derivatized Microparticles and/or Microparticles are biodegradable.

In some embodiments, the molar ratio of dicarboxylic acid to organic polycationic polymer in the second microparticles (i.e., the Microparticles) is 95:5 to 25:75. In some embodiments, the molar ratio of dicarboxylic acid to organic polycationic polymer in the second microparticles (i.e., the Microparticles) is 40:60 to 60:40, more In some embodiments, the molar ratio of dicarboxylic acid to organic polycationic polymer in the second microparticles (i.e., the Microparticles) is around 1:1.

In some embodiments, the total concentration of microparticles (i.e., the Derivatized Microparticles) is 25% to 50% w/v.

In some embodiments, the suspension further comprises a veterinary acceptable carrier. In some embodiments, the veterinary acceptable carrier is chosen from mineral oil, liquid paraffin, white soft paraffin, water, and combinations thereof. In some embodiments, the veterinary acceptable carrier is water.

In some embodiments, the suspension further comprises a gelling agent. In some embodiments the concentration of the gelling agent in the suspension is 0.05% to 5.0% w/v.

In some embodiments, the gelling agent is xanthan gum or sodium carboxymethyl cellulose.

In some embodiments, the veterinary acceptable saturated or unsaturated di-carboxylic acid is chosen from brassylic acid, sebacic acid, azelaic acid, and combinations thereof. In some embodiments, the saturated or unsaturated carboxylic acid is sebacic acid.

In some embodiments, the organic polycationic polymer is chosen from organic polymeric amines, quaternary ammonium compounds, polypeptides, carbohydrates, or combinations thereof. In some embodiments, the organic polycationic polymer is chosen from nisin, ε-polylysine, and chitosan. In some embodiments, the organic polycationic polymer is ε-polylysine.

In some embodiments, the disclosure provides a sterile veterinary formulation comprising a suspension of first microparticles at a concentration of 5% to 60% w/v, wherein the first microparticles are Derivatized Microparticles comprising (i) a veterinary acceptable saturated or unsaturated di-carboxylic acid comprising 8 to 20 carbon atoms, (ii) an organic polycationic polymer, and (iii) an organic polycationic polymer bound to a surface of the first microparticles.

In some embodiments, the sterile veterinary formulation further comprises a suspension of second microparticles, wherein the second microparticles are Microparticles comprising (i) a veterinary acceptable saturated or unsaturated di-carboxylic acid comprising 8 to 20 carbon atoms and an organic polycationic polymer, wherein the total concentration of first and second microparticles in the formulation is 5% to 60% w/v, and wherein the first and second microparticles have a ratio of 90:10 to 10:90 w/w.

In some embodiments, the ratio of first and second microparticles (i.e., Derivatized Microparticles and Microparticles) in the sterile veterinary formulation is 1:1 w/w.

### Definitions

The terms “comprising,” “including,” and “includes” as used herein mean that at least all of the listed elements must be present, but other elements that are not mentioned may also be present.

The term “microparticle” as used herein refers to a particle which has a size on the order of micrometres, such as from 0.05 to 50 μm. In some embodiments, the particle has a size of 0.1 μm to 5 μm. In some embodiments, the particle has a size of 0.1 μm to 1 μm.

The term “Microparticle” as used herein refers to microparticles that comprise (i) a veterinary acceptable saturated or unsaturated di-carboxylic acid that contains eight (8) to twenty (20) carbon atoms and (ii) an organic polycationic polymer. In some embodiments, the Microparticles are obtained by a process comprising contacting the di-carboxylic acid with the polycationic polymer in aqueous solution.

The term “Derivatized Microparticle” as used herein refers to a Microparticle to which an organic polycationic polymer is bound. In some embodiments, the organic polycationic polymer is bound to the external surface of the Microparticle.

The term “external surface” as used herein with respect to Deravitized Microparticles refers to the outer surface of the Derivatized Microparticles, i.e. the surface which faces outward.

The term “polycationic” as used herein means that more than one cationic center and, therefore, more than one positive charge is present. Thus, the term “polycationic polymer” refers to a polymer that has more than one positive charge.

The term “biodegradable” as used herein with respect to microparticles, Microparticles, and Derivatized Microparticles means that the particles can be broken down by naturally occurring enzymes in the environment.

### DETAILED DESCRIPTION

The present disclosure provides compositions and methods for treating and preventing mastitis in cattle by sealing the animal's teats with microparticles that have antimicrobial activity. In some embodiments, the microparticles remain in situ in the lowest part of the teat in the vicinity of streak canal. In some embodiments, the microparticles are Microparticles. In some embodiments, the microparticles are Derivitized Microparticles. In some embodiments, the compositions and methods prevent intra mammary infections in cows during their dry period.

In a first aspect of the disclosure, there is provided a sterile veterinary formulation for use in treating mastitis in cattle, wherein the formulation comprises a suspension of Derivatized Microparticles as herein defined. In some embodiments, the concentration of Derivatzsed Microparticles in the suspension is 5% to 60% on a weight to volume basis (w/v).

In a second aspect of the disclosure, there is provided a method of treating mastitis in cattle, the method comprising administering a sterile veterinary formulation which comprises a suspension of Derivatized Microparticles as herein defined.

In a third aspect of the disclosure, there is provided a sterile veterinary formulation comprising a suspension of Derivatized Microparticles as herein defined and Microparticles as herein defined. In some embodiments, the concentration of Derivatized Microparticles and Microparticles in the suspension is 5% to 60% on a weight to volume basis (w/v).

In some embodiments the organic polycationic polymer bound to the Derivatized Microparticle is an antimicrobial organic polycationic polymer. In yet further embodiments the organic polycationic polymer incorporated in the Microparticle or Derivatized Microparticle is an antimicrobial polycationic polymer.

In a fourth aspect of the disclosure, there is provided a mixture of Derivatized Microparticles as herein defined and Microparticles as herein defined in a ratio of 10:90 to 90:10 w/w. In some embodiments, the Derivatized Microparticles and Microparticles are in a ratio of 40:60 to 60:40 w/w. In some embodiments, the Derivatized Microparticles and Microparticles are in a ratio of 1:1 w/w. In some embodiments, there is provided a veterinary formulation comprising this mixture.

In some embodiments, the suspension comprises 5% to 60% of Derivatized Microparticles and optionally Microparticles on a weight to volume basis (w/v). In some embodiments, the concentration of microparticles in the suspension is 15% to 60% w/v. In some embodiments, the concentration of microparticles in the suspension is 20% to 50% w/v. In some embodiments, the concentration of microparticles in the suspension is 25% to 50% w/v. In some embodiments, the concentration of microparticles in the suspension is 30% w/v.

In some embodiments, the formulation comprises a mixture of Derivatized Microparticles and Microparticles at a concentration of 5% to 60% on a weight to volume basis (w/v). In some embodiments, the ratio of Derivatized Microparticles to Microparticles in the formulation is 90:10 to 10:90 w/w.

In some embodiments, the formulation comprises Derivatized Microparticles to which the organic antimicrobial polycationic polymer is bound in two or more different amounts.

In some embodiments, the formulation additionally comprises a gelling agent. In some embodiments, the gelling agent is chosen from polyethylene oxides, carboxymethyl cellulose, hydroxypropyl methylcellulose, sodium alginate, xanthan gum, and combinations thereof. In some embodiments, the gelling agent is xanthan gum. In some embodiments, the gelling agent is sodium methyl cellulose. In some embodiments, the gelling agent is present in the formulation at concentration of 0.05% to 5.0% on a weight to volume basis (w/v). In some embodiments, the gelling agent is present in the formulation at concentration of 0.3% to 1.2% w/v.

In some embodiments, the formulation further comprises a veterinary acceptable carrier. In some embodiments, the carrier is chosen from mineral oil, liquid paraffin, white soft paraffin, water, and combinations thereof. In some embodiments, the carrier is water. It will be appreciated that the formulations disclosed herein may additionally comprise further components which could be readily identified by the skilled person. For example, the formulations may comprise a thickener, such as aluminium stearate, or a formulation aide, such as colloidal silica.

In some embodiments, the saturated or unsaturated di-carboxylic acid contains eight to twenty carbon atoms. In some embodiments, the saturated or unsaturated di-carboxylic acid contains nine to fifteen carbon atoms. In some embodiments, the saturated or unsaturated di-carboxylic acid contains nine to thirteen carbon atoms. In some embodiments, the di-carboxylic acid is saturated. In some embodiments, the saturated or unsaturated di-carboxylic acid is one which is present in the human/mammalian food chain (such as succinic acid, fumaric acid, malic acid, adipic acid, oxalic acid, sebacic acid, dodecanedioic acid, and amino acids such as aspartic acid and dlutamic acid), or which is already used in human or veterinary medicine. In some embodiments, the saturated or unsaturated di-carboxylic acid is chosen from brassylic acid, sebacic acid, azelaic acid, and combinations thereof. In some embodiments, the di-carboxylic acid is sebacic acid.

Suitable organic polycationic polymers include veterinary acceptable quarternary ammonium compounds, polypeptides, and carbohydrates. Suitable polypeptides include anti-microbial peptides (AMPs) as described in Lei et al (Am J Transl Res 2019; 11 (7); 3919-3931), which is incorporated by reference in its entirety. In some embodiments, the AMP is of the cathelicidin subfamily of AMPs (e.g., Fowlicidin-1, Fowlicidin-2, Fowlicidin-3, LL-37 (hCLD), BMAP-27, Protegrin-3, and Protegrin PG-5). In some embodiments the AMP is of the defensin subfamily (e.g., α-defensin 1, α-defensin 4, α-defensin 5, α-defensin 6, β-defensin 1, β-defensin 2, β-defensin 3, β-defensin 4, antifungal heliomycin, defensin-like peptide-2, and sugarcane defensin 5). In some embodiments, the AMP is bacitracin, dalbavancin, daptomycin, enfuvirtide, oritavancin, teicoplanin, telaprevir, telavancin, or vancomycin. Suitable carbohydrates include chitosan. In some embodiments, the anti-microbial organic polycationic polymers are nisin, and ε-polylysine. In some embodiments, the anti-microbial polycationic polymer is ε-polylysine.

In some embodiments, the Microparticles and Derivatized Microparticles are formed from sebacic acid and ε-polylysine. In some embodiments, the Microparticles and Derivatized Microparticles comprise sebacic acid and ε-polylysine and the Derivatized Microparticles have further ε-polylysine bound to them. In some embodiments, the further ε-polylysine is bound to the external surface of the Derivatized Microparticles. As demonstrated in the Examples, microparticles formed from a combination of sebacic acid and ε-polylysine provide antimicrobial effects and remain in situ in the teat of cattle. They therefore act as an effective teat sealant which provides protection against infection in cattle.

In some embodiments, the organic polycationic polymer contained in the Derivatized Microparticle is the same as the organic antimicrobial polycationic polymer which is bound to the Derivatized Microparticle. In some embodiments, the organic antimicrobial polycationic polymer is ε-polylysine.

In some embodiments, the Microparticles without bound organic antimicrobial polycationic polymer comprise the same saturated or unsaturated dicarboxylic acid and organic polycationic polymer as the Derivatized Microparticles. Thus in some embodiments, the Derivatized Microparticles and Microparticles are both formed from sebacic acid and ε-polylysine.

In some embodiments, the formulation comprises a mixture of Derivatized Microparticles and Microparticles, wherein the ratio of Derivatized Microparticles to Microparticles is 90:10 to 10:90 w/w. In some embodiments, the ratio is 75:25 to 25:75 w/w. In some embodiments, the ratio is 40:60 to 60:40 w/w. In some embodiments, the ratio is 45:55 to 55:45 w/w. In some embodiments, the ratio is 49:51 to 51:49 w/w. In some embodiments, the ratio is 50:50 w/w.

In some embodiments, the Microparticles and Derivatized Microparticles are biodegradable.

In some embodiments, the Microparticles and Derivatized Microparticles are formed in suspension by contacting a long chain fatty dicarboxylic acid with a solution of organic polycationic polymer. This process is described in EP3265139B and U.S. 2018/0036702 A1, which are incorporated by reference in their entirety.

In some embodiments, after forming the Microparticles they are derivatized by bringing them into contact with a solution of the organic antimicrobial polycationic polymer in the presence of a cross-linking agent. In some embodiments, the cross-linking agent is a carbodiimide and or hydroxybenzotriazole. In some embodiments, the cross-linking agent is 1-(3-dimethylaminopropyl)-3-ethylcarbodiimide.

In some embodiments, the Derivatized Microparticles and Microparticles are prepared as dry powders. In some embodiments, the dry powders are prepared from a suspension, for example prepared as above, by spray drying or lyophilization.

In some embodiments, the suspensions of microparticles are formed by suspending the Derivatized Microparticles and Microparticles, if they are present, in a veterinary acceptable carrier, optionally in the presence of a conventional formulation aid. When Microparticles are present, they may be mixed with the Derivatized Microparticles prior to suspending in the veterinary acceptable carrier or by suspending one or both of the Microparticles or Derivatized Microparticles in carriers and then mixing.

In some embodiments, the formulations are administered to the cattle by any suitable means. In some embodiments, the formulation is administered by intra mammary infusion. In some embodiments, the formulation is administered during the cow's dry period. In some embodiments, the formulation is administered via the udder streak canal. Administration during the cow's dry period provides a prophylactic effect whereby future infections associated with milking can be prevented.

In some embodiments, the formulation is administered to the cow in a volume of 1 ml to 10 ml. In some embodiments, the volume is 2 ml to 5 ml. In some embodiments, the formulation comprises a suspension which comprises Derivatized Microparticles in an amount of about 30% on a weight by volume basis (w/v).

### EXAMPLES

### Example 1: Preparation of Microparticles

### (a) (i) Preparation of Microparticles

Microparticles were prepared according to the method of Example 7 of EP 3 265 139 B1, which is incorporated by reference in its entirety. In particular, sebacic acid (14.235 g,) and 4-methylmorpholine (NMM) (15.66 g), were dissolved in water, ε-polylysine (21.889 g,) was dissolved in water and added to the above solution of Sebacic acid/NMM. The mixture was stirred for two hours and then filtered through a 0.5 μm membrane. Dimethylaminopropyl-3-ethylcarbodiimide (3.233 g) and hydroxybenzotriazole (40.468 g) were then added. The mixture was stirred for less than one minute and left overnight. Multilamellar Microparticles had formed.

(Aa) (ii) Preparation of lyophilized Microparticles

The suspension from (a) (i) was subjected to semi-membrane filtration and the product lyophilized to give 14 g of lyophilized Microparticles.

### (b) Preparation of Derivatized Microparticles

ε-polylysine (21.889 g), dimethylaminopropyl-3-ethylcarbodiimide (15.75 g), and hydroxybenzotriazole (1.25 g) were added to the suspension from (a) (i) and the mixture stirred for less than one minute and then left to stand for two hours. The product was filtered through a 53 μm mesh and the resultant suspension subjected to semi-membrane filtration and lyophilization as before to give 14 g of lyophilized Derivatized Microparticles.

### Example 2: Assessment of Bactericidal Activity of Microparticles

Microparticles and Derivatized Microparticles were prepared according to the method in Example 1. A 10% w/v suspension of each of these microparticles was prepared by addition of sterile water and mixing. The bactericidal properties of the two suspensions was compared using an Escherichia coli biofilm assay with sterile water acting as control. Both suspensions were found to have antimicrobial properties and displayed greater bactericidal effects than the control. However, Derivatized Microparticles exhibited greater activity than Microparticles.

### Example 3: Comparison of Antimicrobial Activity of Microparticle Mixtures

The Microparticles and Derivatized Microparticles described in Example 1 were combined in different proportions and suspended to provide two mixtures. In particular, Microparticles were combined with Derivatized Microparticles in ratios of 50:50 and 25:75 w/w to provide a final concentration of 50% w/v in sterile deionised water. The antimicrobial properties of the mixtures were evaluated in the context of Streptococcus uberis, Staphylococcus aureus, and Escherichia coli using standard assays at a commercial microbiology laboratory. Sterile deionised water was included as a control. The bacterial count pre-treatment and after 24 hours of incubation was evaluated in each case and the results are shown in Table 1 below.

TABLE 1
Antibacterial effect of microparticle mixtures with varying
ratios of Derivatized Microparticles and Microparticles

| | Ratio of Microparticles to | Antibacterial effect |
|---|---|---|
| | Derivatized Microparticles | (log reduction in |
| Microrganism | (w/w) | CFU) |
| Streptococcus | 25:75 | 7 |
| uberis | 50:50 | 5 |
| Staphylococcus | 25:75 | 8 |
| aureus | 50:50 | 4 |
| Escherichia coli | 25:75 | 8 |
| | 50:50 | 8 |

Both mixtures were found to be antimicrobial with respect to each microorganism. Of the two mixtures, however, the mixture which contained the greater proportion of Derivatized Microparticles exhibited the greater bactericidal activity.

### Example 4: In Situ Persistence within an In Vitro Udder

The Microparticles and Derivatized Microparticles of Example 1 were combined with water in equal amounts as well as in 75:25 and 25:75 w/w ratios to provide a three different 30% aqueous pastes. An artificial udder was constructed with four blind ending projections below a central reservoir which was open at the top. A 5 ml volume of paste was placed into each teat-like projection after which whole milk was introduced into the reservoir such that contact was made between the paste and the milk. The reservoir was sealed, suspended and gently agitated periodically over three days. Subsequently, the reservoir was drained and the contents of each teat were examined. In all cases the paste was found to remain in situ in the lowest portion of the teat.

### Example 5: In Vivo Challenge Study to Assess Efficacy

25.95 g of each of the Derivatized Microparticles and the Microparticles of Example 1 were suspended and mixed in sterile water until a final volume of 173 ml was achieved to give 30% w/v aqueous paste. The paste was dispensed into 5 ml syringes which were sealed with syringe caps pending use.

Ten cows identified as free of intra mammary pathogens by observation of somatic cell count and by microbiological assessment were dried off. One quarter in each udder was randomly chosen as the control which received no treatment. The other three quarters of each udder were infused via the streak canal with 5 ml of the paste. After two days, five cows were artificially challenged via the streak canal with a well characterised strain of Streptococcus uberis which reliably causes mild infection. A further 5 cows were challenged in a similar manner seven days after infusion with the antimicrobial paste. Duplicate samples of mammary fluid were obtained by manual expression from each quarter two weeks after dry off and these underwent microbiological assessment. Infection with the challenge microorganism was found to be present in all control quarters that had not received paste, but both fluid samples were free of Streptococcus uberis in 13 of the treated quarters despite bacterial challenge. The 100% rate of infection with the challenge microorganism in the control quarters revealed the challenge to be robust and the 43% freedom from Streptococcus uberis in the treated quarters revealed the antimicrobial paste to be capable of preventing intra mammary infection. For context, the published results of a similar challenge study (Twomey D P et. al, “Protection against Staphylococcus aureus mastitis in dairy cows using a bismuth-based teat seal containing the bacteriocin, lacticin”, 3147. J Dairy Sci. 2000 September; 83 (9): 1981-8. doi: 10.3168/jds.S0022-0302 (00) 75075-2. PMID: 11003227, incorporated herein by reference in its entirety) revealed infection to be present in 100% of quarters infused with bismuth subnitrate internal teat sealant mixed with Lacticin 3147 and this indicated failure to provide protection against the bacterial challenge.

### Example 6: Mousse Formation

Syringes of Microparticles and Derivatized Microparticles were prepared according to Example 5. Paste-like suspensions of Derived Microparticles/Microparticles were created by suspending quantities of the microparticles in water. Due to the different properties of the two materials, the suspension concentration varied depending on the composition. The concentrations are shown in Table 2 below. It can be seen that higher concentrations of Derived Microparticles were needed to make a paste compared with Microparticles.

TABLE 2
Concentrations of Derived Microparticles and Microparticles
in each of the paste-like suspensions

| | | | 75:25 w/w | | 50:50 w/w | | | |
|---|---|---|---|---|---|---|---|---|
| | | | Derivatized | | Derivatized | | | |
| | Derivatized | | Microparticles/ | | Microparticles/ | | | |
| | Microparticles | | Microparticles | | Microparticles | | Microparticles | |
| Microparticles | — | | 550 | mg | 1100 | mg | 2200 | mg |
| Derivatized | 2200 | mg | 1650 | mg | 1100 | mg | — | |
| Microparticles | | | | | | | | |
| Water | 5.7 | ml | 4.3 | ml | 6.6 | ml | 9.8 | ml |
| Total volume | ~7.9 | ml | ~6.5 | ml | ~8.8 | ml | ~12 | ml |

Crude artificial cow teats were created using 5 ml syringes, luer lock caps, and O-rings made from plunger rubber. Each was filled with one of the suspensions described above before being placed in the finger of a latex glove. Full fat milk containing blue food colouring was added to provide a milk reservoir above the artificial teat to mimic the gland cistern. The gloves were tied off and suspended from a pole which was gently agitated by being carried over a distance of 2 km to create an artificial udder.

The artificial udders were left undisturbed for 24 hours and the syringes were then removed from the artificial udders and examined. In the cases of the suspensions made entirely from the Microspheres and entirely from the Derived Microspheres, some loss of material from the confines of the syringes was evident. Conversely, for the suspensions made from a combination of the two materials, a stable mousse like interface between the microsphere suspension and the coloured milk was present. The quality and durability of the mousse created with a 50:50 w/w mix of batches was superior to that made with the unbalanced mixture.

### Example 7 Gelling Agents

To improve the stability and viscosity of the formulated suspension, an initial screen of different viscosity modifiers was performed using high and low excipient concentrations. Included in the list of viscosity modifiers were xantham gum, carboxymethyl cellulose, sodium alginate, and poloxamer P188. Assessment of the resulting formulations was achieved subjectively, by consideration of appearance and syringeability, and objectively, by measurement of viscosity using an Anton Paar MCR 302 Rheometer. These assessments were made following 40° C. storage at T=0 and at T=2 weeks.

| | Weight of components before addition of water (mg) | | | | | Total | Viscosity | | | |
|---|---|---|---|---|---|---|---|---|---|---|
| | | Non- | | | | weight of | modifier | % | % Non- | |
| | Derivatized | derivatized | | | Additional | prototype | concen- | Derivatized | derivatized | |
| | micro- | micro- | Viscosity | | water | formulation | tration | micro- | micro- | |
| Prototype | particles | particles | modifier | Water | (mg) | (mg) | (%) | particles | particles | Water |
| Xanthan | 543.51 | 543.64 | 12.49 | 1384.26 | 650 | 3133.9 | 0.4 | 17.34 | 17.35 | 64.91 |
| 0.5% | | | | | | | | | | |
| Xanthan | 531.7 | 531.96 | 37.27 | 1384.34 | 550 | 3035.27 | 1.2 | 17.52 | 17.53 | 63.73 |
| 1.5% | | | | | | | | | | |
| CMC | 544.4 | 544.59 | 12.48 | 1381.69 | 650 | 3133.16 | 0.4 | 17.38 | 17.38 | 64.84 |
| 0.5% | | | | | | | | | | |
| CMC | 531.74 | 530.69 | 37.34 | 1388.95 | 900 | 3388.72 | 1.1 | 15.69 | 15.66 | 67.55 |
| 1.5% | | | | | | | | | | |
| Alginate | 543.59 | 543.82 | 12.35 | 1392 | 900 | 3391.76 | 0.4 | 16.03 | 16.03 | 67.58 |
| 0.5% | | | | | | | | | | |
| Alginate | 532.25 | 531.62 | 37.27 | 1383.69 | 900 | 3384.83 | 1.1 | 15.72 | 15.71 | 67.47 |
| 1.5% | | | | | | | | | | |
| Polox 1% | 537.75 | 537.85 | 24.81 | 1383.98 | 650 | 3134.39 | 0.8 | 17.16 | 17.16 | 64.89 |
| Polox 5% | 487.92 | 487.64 | 124.4 | 1389.52 | 400 | 2889.48 | 4.3 | 16.89 | 16.88 | 61.93 |

The best viscosity modifiers for inclusion in the formulation that provided the desired properties under the conditions of assessment were xanthan gum at 0.4% and sodium carboxymethyl cellulose at 1.1%. These formulations demonstrated consistent viscosity, maintained appearance, and were syringeable under the testing conditions, making them the most suitable candidates for further formulation development and stability studies.

The present disclosure provides exemplary embodiments and is not intended to be limiting. It will be appreciated that various other modifications and variations are also possible.

1. A method for treating or preventing mastitis in cattle, comprising administering to the cattle an effective amount of a suspension comprising a plurality of first microparticles at a concentration of 5% to 60% w/v, wherein the first microparticles comprise (i) a veterinary acceptable saturated or unsaturated di-carboxylic acid comprising 8 to 20 carbon atoms, (ii) an organic polycationic polymer, and (iii) an organic polycationic polymer bound to a surface of the first microparticles.

2. The method according to claim 1, wherein the suspension is administered during the dry period of the cow.

3. The method of claim 1, wherein the polycationic polymer in (ii) and (iii) is antimicrobial.

4. The method according to claim 2, wherein the suspension further comprises a plurality of second microparticles comprising (i) a veterinary acceptable saturated or unsaturated di-carboxylic acid comprising 8 to 20 carbon atoms and (ii) an organic polycationic polymer, wherein the first and second microparticles have a combined concentration in the suspension of 5% to 60% w/v, and wherein the first and second microparticles have a ratio of 90:10 to 10:90 w/w in the suspension.

5. The method according to claim 4, wherein the polycationic polymer in the second microparticles is antimicrobial.

6. The method according to claim 1, wherein the microparticles are biodegradable.

7. The method according to claim 1, wherein the dicarboxylic acid and organic polycationic polymer have a molar ratio of 95:5 to 25:75 in the second microparticles.

8. The method of claim 1, wherein the total concentration of microparticles in the suspension comprises 25% to 50% w/v.

9. The method according to claim 1, wherein the suspension further comprises a veterinary acceptable carrier chosen from mineral oil, liquid paraffin, white soft paraffin, water, and combinations thereof.

10. The method according to claim 9, wherein the veterinary acceptable carrier is water.

11. The method according to claim 1, wherein the suspension further comprises a gelling agent.

12. The method according to claim 11, wherein the gelling agent is chosen from xanthan gum and sodium carboxymethyl cellulose and has a concentration of 0.05% to 5.0% w/v.

13. The method according to claim 1, wherein the veterinary acceptable saturated or unsaturated di-carboxylic acid is chosen from brassylic acid, sebacic acid, azelaic acid, and combinations thereof.

14. The method according to claim 13, wherein the saturated or unsaturated carboxylic acid is sebacic acid.

15. The method according to claim 1, wherein the organic polycationic polymer is chosen from organic polymeric amines, quaternary ammonium compounds, polypeptides, carbohydrates, or combinations thereof.

16. The method according to claim 15, wherein the organic polycationic polymer is chosen from nisin, ε-polylysine, and chitosan.

17. The method according to claim 16 wherein the organic polycationic polymer is ε-polylysine.

18. A sterile veterinary formulation comprising a suspension of first microparticles at a concentration of 5% to 60% w/v, wherein the first microparticles comprise (i) a veterinary acceptable saturated or unsaturated di-carboxylic acid comprising 8 to 20 carbon atoms, (ii) an organic polycationic polymer, and (iii) an organic polycationic polymer bound to a surface of the first microparticles.

19. The sterile veterinary formulation according to claim 18, further comprising a suspension of second microparticles comprising (i) a veterinary acceptable saturated or unsaturated di-carboxylic acid comprising 8 to 20 carbon atoms and an organic polycationic polymer, wherein the first and second microparticles have a combined concentration in the formulation of 5% to 60% w/v, and wherein the first and second microparticles have a ratio of 90:10 to 10:90 w/w.

20. The sterile veterinary formulation of claim 19, wherein the ratio of first and second microparticles is 1:1 w/w.
